(19) United States
(12) Patent Application Publication
(10) Pub. No.: US 2022/0084623 A1
(43) Pub. Date: Mar. 17, 2022

(54) Method for analyzing relationship between communication path and heat resistance of lipase

(71) Applicant: Jiangnan University, Wuxi (CN)

(72) Inventors: Yanrui DING, Wuxi (CN); Qian XIA, Wuxi (CN)

(21) Appl. No.: 17/534,542

(22) Filed: Nov. 24, 2021

Related U.S. Application Data
(63) Continuation of application No. PCT/CN2020/070784, filed on Jan. 8, 2020.

(30) Foreign Application Priority Data
Jun. 19, 2019 (CN) 201910529517.2

(51) Int. Cl. G16B 5/30; G16B 15/20

(52) U.S. Cl. CPC G16B 5/30; G16B 15/20

(57) ABSTRACT

The present disclosure herein discloses a method for analyzing a relationship between a communication path and heat resistance of lipase, and belongs to the technical field of computer application. The relationship between the dynamic communication and the thermal stability of the lipase is revealed conveniently and fast by combining a Dijkstra algorithm and an Apriori-like algorithm, path information between any two residues inside a structure is mined, a problem that in the prior art, an important path segment inside is not mined and important path information tends to be ignored is solved, and an effect of more accurately demonstrating difference of communication strength between residues is achieved; and besides, the revealed relationship between the communication and the thermal stability of the lipase in the application is dynamic, a defect that only a relationship between static communication and the thermal stability of the lipase is reflected in the prior art is overcome, and an effective means is provided for studying the thermal stability of a protein in whole from an overall perspective.

### TECHNICAL FIELD

The present disclosure herein relates to a method for analyzing a relationship between a communication path and heat resistance of lipase, and belongs to the technical field of computer application.

### BACKGROUND

In over a decade, with development of a complex network theory, a protein structure is encoded into a residue interaction network for analyzing a relationship between the protein structure and its function. The residue interaction network can study a relationship among a protein spatial structure, protein properties and its function from a system perspective and can be used not only for discovering a topological characteristic of the protein structure, but also for exploring a relationship between protein dynamics and signal transduction, etc. A communication path is an important characteristic between two residues in the residue interaction network, and recognizing change of dynamic communication between residues in lipase based on the residue interaction network can promote research of a relationship between protein communication and thermal stability. At present, there is few research methods for studying communication in a dynamic residue interaction network and combining the communication of the dynamic residue interaction network and thermal stability of the lipase, and thus, a research method is to be invented so as to further facilitate research of the relationship between the protein structure and its function.

Raimondi F et al. (Light on the structural communication in Ras GTPases. Journal of Biomolecular Structure and Dynamics, 2013, 31(2): 142-157.) combine a graph theory with fluctuation dynamics in a frame of protein structure network analysis to explore structural communication of a protein, a frequency of appearance of an appearing path within simulation time is statistically calculated simply after a shortest path is determined, however, an important path segment inside is not discovered, and therefore, it tends to ignore important path information if this method is combined with thermal stability research. James K A et al. (Structure-based network analysis of activation mechanisms in the ErbB family of receptor tyrosine kinases: the regulatory spine residues are global mediators of structural stability and allosteric interactions. PLoS One, 2014, 9(11): e113488.) study allosteric communication of a protein structure network, high-centrality residues in information transfer in the network are recognized by calculating network betweenness, it is discovered that these high-centrality residues play an important role in communication in a catalytic domain, however, simple betweenness calculation cannot help to capture a change process of communication among all the residues. Ribeiro A A S T et al. (Determination of signaling pathways in proteins through network theory: importance of the topology. Journal of chemical theory and computation, 2014, 10(4): 1762-1769.) establish the protein structure network by calculating interaction energy among the residues, and a signal propagation path among binding sites is recognized based on a network topological structure. However, this method only simply considers the network topological structure while recognizing a communication path without combining strength of edge connection among the residues, and consequently, difference of communication strength among the residues cannot be demonstrated accurately.

A Dijkstra algorithm is an algorithm for calculating a shortest path from one top vertex to other top vertexes and is mainly characterized by extending outwards layer by layer with an initial point as a center till it reaches a terminal point. An Apriori algorithm is a frequent item-set algorithm for mining association rules and is repeatedly improved into an Apriori-like algorithm and widely applied to various fields such as logistics, business and network security. However, there is no report of combining and applying the Dijkstra algorithm and the Apriori-like algorithm to explore a relationship between communication and thermal stability in the dynamic residue interaction network of the lipase in documents and patents.

### SUMMARY

In order to reveal a relationship between dynamic communication and thermal stability of lipase and mine path information between any two residues inside a structure, the present disclosure herein provides a method for analyzing a relationship between a communication path and heat resistance of lipase in a dynamic residue interaction network. The method includes: working out, based on the residue interaction network, a dynamic shortest communication path between residues through a Dijkstra algorithm, and then performing frequent path mining on the shortest communication path through an Apriori-like algorithm so as to obtain a rigid communication path of the lipase and a relationship between the rigid communication path and thermal stability of the lipase, wherein directed sequentiality of the path and a topological characteristic of the network are considered while performing frequent path mining on the shortest communication path through the Apriori-like algorithm.

Alternatively, the method includes:

S1: extracting a three-dimensional structure of the lipase and its mutant from a PDB, and performing molecular dynamics simulation based on an Amber force field so as to obtain a molecular motion trajectory file of the lipase within simulation time at different temperatures; and intercepting N frames of snapshots within the simulation time, where N is an integer;

S2: establishing N frames of weighting residue interaction networks according to the molecular motion trajectory file obtained in the S1;

S3: calculating a shortest path between an initial residue Rₛ and a terminal residue Rₜ in the N frames of weighting residue interaction networks on the basis of the Dijkstra algorithm, where s≠t, s, t∈{1,2, . . . ,n};

S4: performing frequent path mining based on a time sequence on a shortest path between any every two residues through the Apriori-like algorithm; and

S5: performing, through the Apriori-like algorithm, frequent path mining on a time sequence frequent path or path segment mined in the S4 so as to obtain the rigid communication path of the lipase and then obtain the relationship between the rigid communication path and the thermal stability of the lipase.

Alternatively, the S2 includes:

encoding a structure of the lipase in each frame of snapshot intercepted within the simulation time in the S1 into a residue interaction network changing over time; and extracting interaction force between the residues in the molecular motion trajectory file and establishing a static weighting residue interaction network within the whole simulation time according to the interaction force between the residues in each frame of snapshot through Ring2.0.

Alternatively, the S3 includes:

calculating the shortest path between the initial residue R_{S} and the terminal residue Rₜ (s≠t, s, t∈{1,2, . . . ,n}) in each frame of residue interaction network through the Dijkstra algorithm, where n represents the quantity of residues in the lipase, including the following steps:

step 1: initializing distances between the initial residue Rₛ and all the other residues; recording a distance D (Rₛ, Rᵢ) to be a length of an edge between Rₛ and Rᵢ if a direct connection exists between the initial residue Rₛ and Rᵢ; or otherwise, recording the distance D (Rₛ, Rᵢ) to be infinitely great;

step 2: setting two sets U and V, where the set U is configured to sequentially store discovered residues appearing in the shortest path, and at first, the set U contains only the initial residue Rₛ, and the remaining uncertain residues are stored in the set V;

step 3: comparing D (Rₛ, Rᵢ) with D (Rₛ, Rⱼ), where Rᵢ and Rⱼ are the residues in V having direct connection with the initial residue Rₛ, the residue Rₖ having a shortest distance from Rₛ is transferred into the set U, and i, j, k∈{1,2, . . . ,n} and i≠j;

step 4: calculating a distance between the residue Rₖ newly added into U and the residue in the set V having direct connection with Rₖ; recording a distance between Rₛ and Rₘ to be D (Rₛ, Rₖ)+D (Rₖ, Rₘ) if D (Rₛ, Rₘ)>D (Rₛ, Rₖ)+D (Rₖ, Rₘ), where Rₘ∈V; and then putting the residue in V having the shortest distance from Rₛ in U; and

step 5: repeating the step 4 till the terminal residue Rₜ appears in the set U.

Alternatively, the S4 includes:

performing frequent path mining on the shortest path between any every two residues based on the time sequence, and defining a mined frequent path as the time sequence frequent path, where two characteristics of the path, namely, the directed sequentiality of the path and the topological characteristic of the network need to be considered emphatically while mining a frequent path sequence through the Apriori-like algorithm; and

performing time sequence frequent path mining on the shortest path of all residue pairs within the simulation time on the basis of the Apriori-like algorithm, including the following steps:

step 1: establishing a matrix-based data model: storing the shortest path between the same pair of residues Rₛ and Rₜ at different moments according to a time sequence, sequentially recording the residues in each path to be 1, 2, 3, . . . i according to a signal transmission sequence; marking with 0 if there is no residue passed by; and only needing to judge whether a value to which communication residues correspond is an arithmetic progression with a tolerance being 1 when whether rows in a matrix contain a path sequence is judged, where the communication residues are the residues contained in each path;

step 2: generating a candidate frequent 1-item set, where a residue in each item set in a candidate frequent path set C₁ is a starting point of a candidate frequent path; and calculating a support degree Sup of each item set in C₁, and the candidate frequent path is regarded as a frequent path and stored in a set L₁ if Sup is larger than a minimum support threshold value, where a calculation method of the support degree Sup is as follows:

$$S u p = \frac{count}{T} ,$$

where count represents the number of times of appearance of a path segment between Rₛ and Rₜ within the simulation time under observation, and T represents the simulation time;

step 3: connecting the residues in L₁ mutually so as to generate a candidate frequent 2—item set C₂, and then calculating a support degree Sup of each candidate frequent 2-item set in generated C₂, where the calculation method of Sup is the same as that of the step 2; and regarding that the candidate frequent 2—item set is frequent and stored in L₂ if Sup is larger than a set time minimum support degree threshold value; and

step 4: continuing the step 3 till a maximum frequent path sequence Lₚ is discovered.

Alternatively, the time minimum support degree threshold value is 0.5.

Alternatively, in the step 4, the quantity of candidate sets is reduced by using an Apriori attribute, namely, all nonempty subsequences of any frequent sequence set are frequent necessarily, otherwise, this candidate frequent path sequence will be removed from Cₚ, and Cₚ is a set of candidate frequent paths with all path lengths being p-1.

Alternatively, in the S5, a space-time frequent path means a time sequence frequent path or path segment frequently appearing in paths with different starting points and end points and represents information of a shared communication path between the residues, and thus, the space-time frequent path reflects communication characteristics of the residues from an overall perspective. Before performing space-time frequent path mining, all time sequence frequent path p sequences of each pair of residues in the matrix are used as a data set, a space-time minimum support degree threshold value is set, the space-time frequent path is mined, and thus a relationship between the heat resistance of the lipase and signal transfer is explored, where p is a length of a longest time sequence frequent path and is not smaller than 4.

Alternatively, the space-time minimum support degree threshold value is 0.2.

A second objective of the present disclosure herein is to provide application of the method for analyzing the relationship between the communication path and the heat resistance of the lipase in the dynamic residue interaction network in a field of the protein structure and function.

The present disclosure herein has the beneficial effects:

the relationship between the dynamic communication and the thermal stability of the lipase is revealed conveniently and fast by combining the Dijkstra algorithm and the Apriori-like algorithm, the path information between any two residues inside the structure is mined, a problem that in the prior art, an important path segment inside is not mined and important path information tends to be ignored is solved, and an effect of more accurately demonstrating the difference of the communication strength between the residues is achieved. Besides, the revealed relationship between the communication and the thermal stability of the lipase in the application is dynamic, a defect that only a relationship between static communication and the thermal stability of the lipase is reflected in the prior art is overcome, the method of the present disclosure herein combines the shortest path Dijkstra algorithm and the Apriori-like algorithm and applies them to the research of the relationship of the thermal stability of the lipase for the first time, which develops a new thought for the research of the protein thermal stability and meanwhile provides a new method for the research of the relationship between dynamic communication and thermal stability of the protein.

### BRIEF DESCRIPTION OF FIGURES

In order to more clearly illustrate technical schemes of the examples of the present disclosure herein, the accompanying drawings used in the description of the examples are briefly introduced below. It is obvious that the accompanying drawings in the following description are only some examples of the present disclosure herein, and other accompanying drawings may be obtained by those ordinarily skilled in the art based on these accompanying drawings without any creative effort.

FIG. 1 is a diagram of shortest paths within simulation time by taking Ala15 to Ser17 in WTL for example in an example of the present disclosure herein.

FIG. 2 is a diagram of the quantity of residue pairs with large fluctuation of shortest paths and the quantity of residue pairs capable of maintaining stable communication in statistical WTL and 6B in an example of the present disclosure herein.

FIG. 3A, FIG. 3B, FIG. 3C, FIG. 3D, FIG. 3E, FIG. 3F, FIG. 3G, FIG. 3H are a schematic diagram of part of results of a space-time frequent path in an example of the present disclosure herein.

### DETAILED DESCRIPTION

In order to make objectives, technical schemes and advantages of the present disclosure herein clearer, implementations of the present disclosure herein will be further described in detail below in combination with the drawings.

### Example 1

The example provides a method for analyzing a relationship between a communication path and heat resistance of lipase in a dynamic residue interaction network. In the method, on the basis of the residue interaction network, a dynamic shortest communication path between residues is worked out by using a Dijkstra algorithm and then frequent path mining is performed on the shortest communication path through an Apriori-like algorithm so as to obtain a rigid communication path of the lipase and then obtain a relationship between the rigid communication path and thermal stability of the lipase, where directed sequentiality of the path and a topological characteristic of the network are considered while frequent path mining is performed on the shortest communication path through the Apriori-like algorithm.

Specifically, the method includes the following steps.

S1: a three-dimensional structure of the lipase and its mutant is extracted from a PDB, molecular dynamics simulation is performed based on an Amber force field so as to obtain a molecular motion trajectory file of the lipase within simulation time at different temperatures; and intercepting N frames of snapshots within the simulation time, where N is an integer.

S2: N frames of weighting residue interaction networks are established according to the molecular motion trajectory file obtained in the S1.

A structure of the lipase in each frame of snapshot intercepted within the simulation time in the S1 is encoded into a residue interaction network changing over time; and interaction force between the residues in the molecular motion trajectory file is extracted and a static weighting residue interaction network within the whole simulation time is established according to the interaction force between the residues in each frame of snapshot through Ring2.0.

S3: a shortest path between an initial residue Rₛ and a terminal residue Rₜ in the N frames of weighting residue interaction networks is calculated on the basis of the Dijkstra algorithm, where s≠t, s, t∈{1,2, . . . ,n}.

The shortest path between the initial residue Rₛ and the terminal residue Rₜ(s≠t, s, t∈{1,2, . . . ,n}) in each frame of residue interaction network is calculated through the Dijkstra algorithm, where n represents the quantity of residues in the lipase, including the following steps.

Step 1: distances between the initial residue Rₛ and all the other residues are initialized; a distance D (Rₛ, Rᵢ) is recorded to be a length of an edge between Rₛ and Rᵢ if a direct connection exists between the initial residue Rₛ and Rᵢ; or otherwise, D (Rₛ, Rᵢ) is recorded to be infinitely great.

Step 2: two sets U and V are set, where the set U is configured to sequentially store discovered residues appearing in the shortest path, and at first, the set U contains only the initial residue Rₛ, and the remaining uncertain residues are stored in the set V.

Step 3: D (Rₛ, Rᵢ) and D (Rₛ, Rⱼ) are compared, where Rᵢ and Rⱼ are the residues in the set V having direct connection with the initial residue Rₛ, the residue Rₖ having a shortest distance from Rₛ is transferred into the set U, and i, j, k∈{1,2, . . . ,n} and i≠j.

Step 4: a distance between the residue Rₖ newly added into U and the residue in the set V having direct connection with Rₖ is calculated; a distance between Rₛ and Rₘ is recorded to be D (Rₛ, Rₖ)+D (Rₖ, Rₘ) if D (Rₛ, Rₘ)>D (Rₛ, Rₖ)+D (Rₖ, Rₘ), where Rₘ^{∈}V; and then the residue in the set V having the shortest distance from Rₛ is put in U.

Step 5: the step 4 is repeated till the terminal residue Rₜ appears in the set U.

S4: frequent path mining based on a time sequence is performed on a shortest path between any every two residues through the Apriori-like algorithm; and

frequent path mining is performed on the shortest path between any every two residues based on the time sequence, a mined frequent path is defined as the time sequence frequent path, where two characteristics of the path, namely, the directed sequentiality of the path and the topological characteristic of the network need to be considered emphatically while a frequent path sequence is mined through the Apriori-like algorithm.

The step that time sequence frequent path mining is performed on the shortest path of all residue pairs within the simulation time on the basis of the Apriori-like algorithm includes the following steps.

Step 1: a matrix-based data model is established: the shortest path between the same pair of residues Rₛ and Rₜ at different moments is stored according to a time sequence, the residues in each path are sequentially recorded to be 1, 2, 3, . . . i according to a signal transmission sequence; marking is made with 0 if there is no residue passed by; and it is only needed to judge whether a value to which communication residues correspond is an arithmetic progression with a tolerance being 1 when whether rows in a matrix contain a path sequence is judged, where the communication residues are the residues contained in each path, and the data model is shown in table 1.

TABLE 1
Shortest path of Rₛ and Rₜ over time

| Time frame | Rₐ | R_{b} | R_{c} | R_{d} | Rₑ | R_{f} | R_{g} | Rₛ | Rₜ |
|---|---|---|---|---|---|---|---|---|---|
| t₁ | 2 | 4 | 5 | 0 | 3 | 0 | 0 | 1 | 6 |
| t₂ | 0 | 0 | 0 | 0 | 3 | 2 | 4 | 1 | 5 |
| t₃ | 0 | 0 | 0 | 5 | 3 | 2 | 4 | 1 | 6 |
| t₄ | 0 | 0 | 0 | 0 | 3 | 2 | 4 | 1 | 5 |
| t₅ | 0 | 2 | 3 | 5 | 0 | 0 | 4 | 1 | 6 |
| . . . | | | | | . . . | . . . | | | |
| t₃₀ | 0 | 3 | 4 | 0 | 0 | 2 | 0 | 1 | 5 |

Step 2: a candidate frequent 1-item set is generated, where a residue in each item set in a candidate frequent path set C₁ is a starting point of a candidate frequent path; and a support degree Sup of each item set in C₁ is calculated, and the candidate frequent path is regarded as a frequent path and stored in a set L₁ if Sup is larger than a minimum support threshold value, where a calculation method of the support degree Sup is as follows:

$$S u p = \frac{count}{T} ,$$

where count represents the number of times of appearance of a path segment between Rₛ and Rₜ within the simulation time under observation, and T represents the simulation time.

Step 3: the residues in L₁ are connected mutually so as to generate a candidate frequent 2—item set C₂, and then a support degree Sup of each candidate frequent 2—item set in generated C₂ is calculated, where the calculation method of Sup is the same as that of step 2; and it is regarded that the candidate frequent 2—item set is frequent and stored in L₂ if Sup is larger than a set time minimum support degree threshold value, where the time minimum support degree threshold value is 0.5.

Step 4: the step 3 is continued till a maximum frequent path sequence Lₚ is discovered. The quantity of candidate sets may be reduced by using an Apriori attribute, namely, all nonempty subsequences of any frequent sequence set are frequent necessarily, otherwise, this candidate frequent path sequence will be removed from Cₚ, and Cₚ is a set of candidate frequent paths with all path lengths being p-1.

S5: frequent path mining is performed on the time sequence frequent path or path segment mined in the S4 through the Apriori-like algorithm so as to obtain the rigid communication path of the lipase and then obtain the relationship between the rigid communication path and the thermal stability of the lipase.

A space-time frequent path means a time sequence frequent path or path segment frequently appearing in paths with different starting points and end points and represents information of a shared communication path between the residues, and thus, the space-time frequent path reflects communication characteristics of the residues from an overall perspective. Before space-time frequent path mining is performed, all time sequence frequent path p sequences of each pair of residues in the matrix are used as a data set, a space-time minimum support degree threshold value is set, the space-time frequent path is mined, and thus a relationship between the heat resistance of the lipase and signal transfer is explored, where p is a length of a longest time sequence frequent path and is not smaller than 4.

Data used in the example of the present disclosure herein are from a crystal structure of a RCSB PDB (Program Database File) (http://www.rcsb.org/pdb/home/home.do), a wild Type WTL (PDB:1l6W) and its mutant 6B (PDB:3QMM) from Bacillus subtilis lipase are selected as an experimental subject, and N is 179.

(1) 300 ns molecular dynamics trajectory of WTL and 6B at two different simulation temperatures of 300 K and 400 K is obtained through MD simulation, and 3000 frames of snapshots are intercepted within the simulation time.

(2) A weighting residue interaction network is established for each frame of snapshot, its specific process is: a hydrogen bond, a salt bridge and a disulfide bond are calculated with 4 Å as an interatomic distance, a van der Waals force is calculated with 0.8 Å as a distance standard, a π-π action force and a π-cation action force are calculated with 7.0 Å as a distance standard, an interaction force between the residues is calculated with Cα of the residues as a center, a weight of edge is the quantity of all interactions, and whole calculation is realized through Ring2.0.

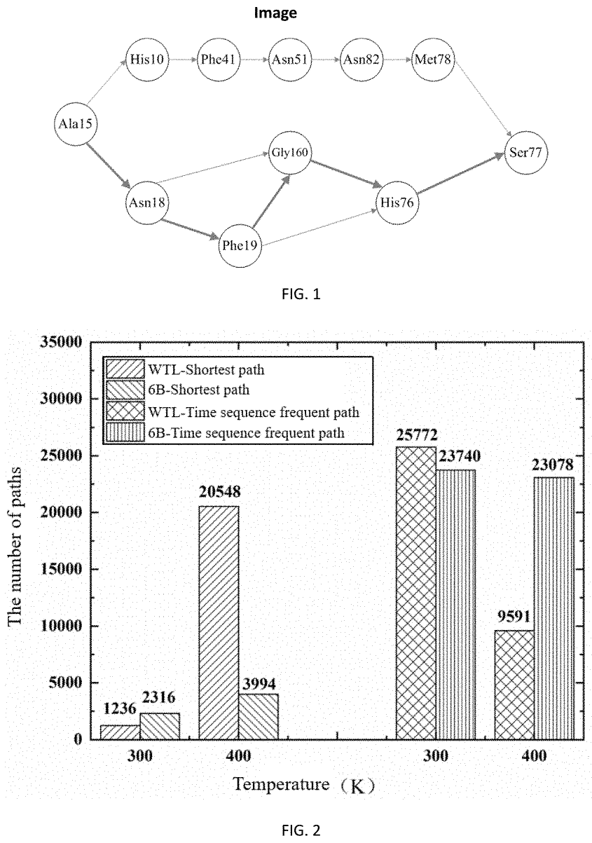

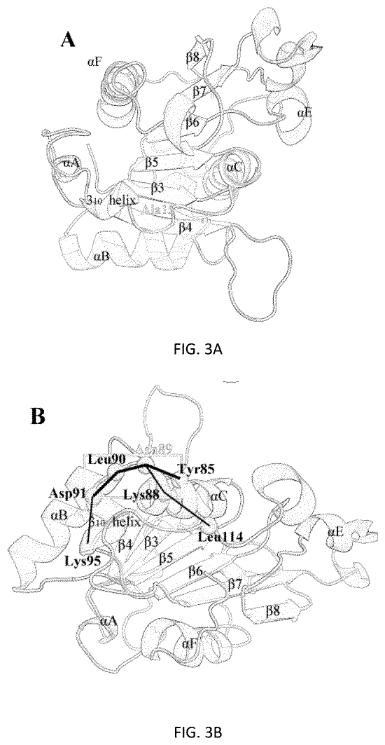

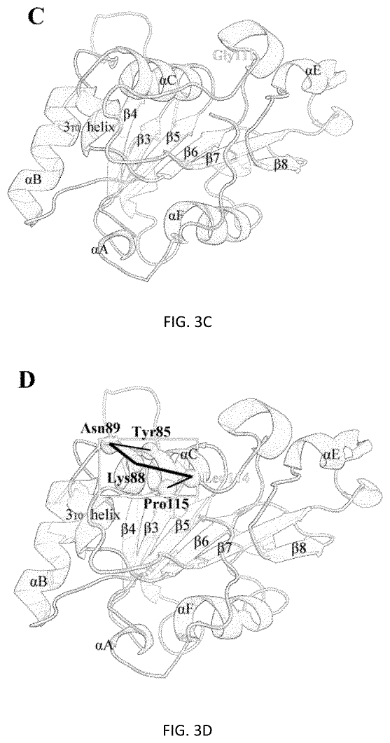

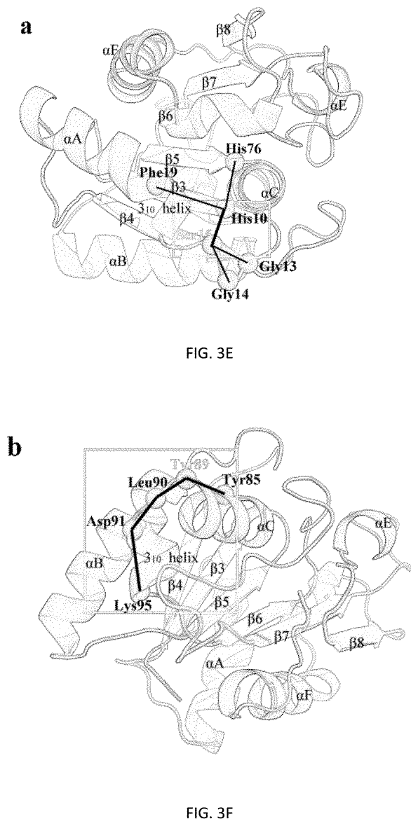

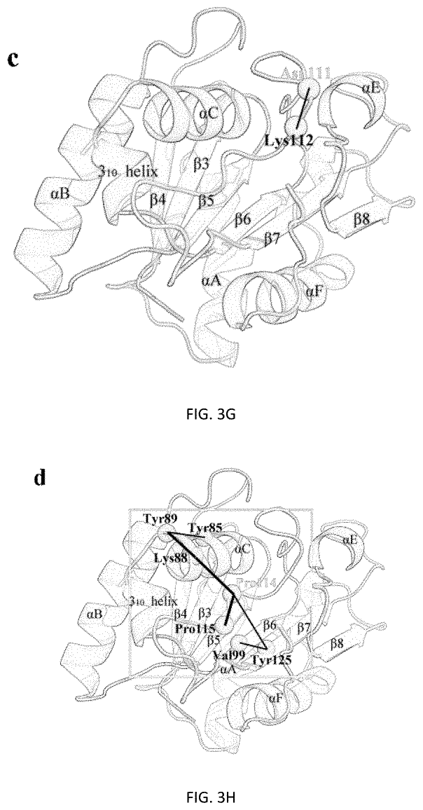

(3) shortest paths between all residue pairs in WTL and 6B under 300 K and 400 K are calculated respectively through the Dijkstra algorithm, and an experimental result example is shown in FIG. 1. Then time sequence frequent path mining is performed on the shortest path between each pair of residues on the basis of the Apriori-like algorithm, a communication difference of WTL and 6B are analyzed from a local perspective, experimental data are shown in FIG. 2, then the space-time frequent path is mined according to the time sequence frequent path by using the Apriori-like algorithm, finally, a space-time frequent path difference in WTL and 6B is analyzed from the overall perspective so as to obtain the relationship between the communication and the heat resistance of the lipase, and part of an experimental result is shown in FIG. 3A, FIG. 3B, FIG. 3C, FIG. 3D, FIG. 3E, FIG. 3F, FIG. 3G and FIG. 3H.

FIG. 1 shows shortest paths of Ala15 to Ser77 in WTL within simulation time. In figure, thick and thin different line segments roughly represent path segments rows pass by at different moments, which means that communication between the residues change dynamically over time and shows that the Dijkstra algorithm can effectively capture dynamic change of the communication between the residues of the lipase.

FIG. 2 is a diagram of the quantity of residue pairs with large fluctuation of shortest paths and the quantity of residue pairs capable of maintaining stable communication in statistical WTL and 6B after time sequence frequent path mining. It can be seen from the figure that WTL and 6B differ obviously.

FIG. 3A, FIG. 3B, FIG. 3C, FIG. 3D, FIG. 3E, FIG. 3F, FIG. 3G, FIG. 3H are a schematic diagram of part of results of a space-time frequent path, which shows the space-time frequent communication paths of WTL and 6B in the paths of A15S, N89Y, G111D and L114P under 400 K. The difference of the rigid communication of WTL and 6B in space and time can be discovered.

FIG. 2 and FIG. 3A, FIG. 3B, FIG. 3C, FIG. 3D, FIG. 3E, FIG. 3F, FIG. 3G and FIG. 3H comprehensively illustrate that the Apriori-like algorithm fused with path characteristics can effectively mine characteristics of dynamic communication of the lipase and provide a new research thought and method for studying a relationship between dynamic communication and the thermal stability of protein.

In the drawings, FIG. 1: shortest paths of Ala15 to Ser77 in WTL within simulation time, which include four paths:

Ala15-His10-Phe41-Asn51-Asn82-Met78-Ser77,

Ala15-Asn18-Phe19-Gly160-His76-Ser77,

Ala15-Asn18-Gly160-His76-Ser77, and

Ala15-Asn18-Phe19-His76-Ser77. In FIG. 1, the thicker the line segments are, the more frequent the path segments are.

FIG. 2: the quantity of residue pairs with large fluctuation of shortest paths and the quantity of residue pairs capable of maintaining stable communication in WTL and 6B.

FIG. 3A, FIG. 3B, FIG. 3C, FIG. 3D, FIG. 3E, FIG. 3F, FIG. 3G, FIG. 3H: a diagram of space-time frequent communication of WTL and 6B in paths of A15S, N89Y, G111D and L114P under 400 K. In a WTL structure, the space-time frequent paths are: FIG.A none, FIG.B Tyr85-Asn89-Lys88-Leu114, Tyr85-Asn89-Leu90-Asp91, Asn89-Leu90-Asp91-Lys95, FIG.C none and FIG.D Tyr85-Asn89-Lys88-Leu114, Asn89-Lys88-Leu114-Pro115. In a 6B structure, the space-time frequent paths are: FIG. E Gly13-Ser15-His10, Gly14-Ser15-His10, Ser15-His10-Phe19, Ser15-His10-His76, FIG. F Tyr85-Tyr89-Leu90-Asp91-Lys95, Lys95-Asp91-Leu90-Tyr89-Tyr85, FIG. G Asp111-Lys112 and FIG. H Tyr85-Tyr89-Lys88-Pro114, Tyr89-Lys88-Pro114-Pro115, Val99-Tyr125-Pro114-Pro115.

The method of the present application has the advantages that: 1. more path segment information is mined, and 2. the mined information is dynamic.

The following is a comparison of the method of the present application with several other methods for studying protein network communication in terms of advantages.

Raimondi F et al. (Light on the structural communication in Ras GTPases. Journal of Biomolecular Structure and Dynamics, 2013, 31(2): 142-157.) selects an analysis subject by using a frequency of appearance of a whole path within whole simulation time as a standard when analyzing structural communication path characteristics of Ras GTPases. By means of this method, a whole path with higher frequency of appearance may be rapidly found, however, important communication paths of small segments contained in the path intend to be ignored, and actually, a long path with a lower frequency of appearance may contain a short path with a higher frequency of appearance. By means of the method of the present application, the directed sequentiality characteristic of the path is combined with steps of the Apriori algorithm, frequencies of the path segments from shorter ones to longer ones in each path are calculated, and finally, the communication path playing an important role is mined. In this way, it is beneficial for guaranteeing that information of each small path segment is not ignored. Thus, compared with the method of Raimondi et al., the method of the present application can mine communication characteristics of more path segments.

James K A et al. (Structure-based network analysis of activation mechanisms in the ErbB family of receptor tyrosine kinases: the regulatory spine residues are global mediators of structural stability and allosteric interactions. PLoS One, 2014, 9(11): e113488.) study communication in a protein structure network by calculating a node betweenness value of residues. A node betweenness is a ratio of the quantity of shortest paths passing by a node among all shortest paths in the network to the total quantity of shortest paths and can reflect communication significance of one node in the network, however, this method only simply calculates communication significance of each node in a static network and cannot demonstrate dynamic change of communication between the residues in network over time. It is well known that an amino acid network will change along with passage of time instead of being fixed when affected by an external factor, so that focusing on only static communication is not conform to the actual. The method of the present application calculates a shortest path between any pair of residues in the amino acid network at every moment and mines important communication path segments with higher frequencies of appearance between the residues from two dimensions of space and time, and as the communication path with the higher frequency of appearance usually reflects stable connection between protein structures, a role of these important communication path segments played in structural stability of the lipase under high temperature are finally discovered. Therefore, compared with the method of James K A et al., the method of the present application can discover dynamic change of communication between the residues in the amino acid network and is more suitable for analyzing the relationship between the dynamic communication between the residues and the thermal stability of the protein.

Ribeiro A A S T et al. (Determination of signaling pathways in proteins through network theory: importance of the topology. Journal of chemical theory and computation, 2014, 10(4): 1762-1769.) adopts a method for recognizing a signal propagation path between binding sites based on a network topological structure, however, it believes that there is communication if there is connection between the residues, has the same communication strength and does not consider that actually, influences on the communication strength between every two residues are different due to their interaction though it can determine a communication path between the residues. Therefore, this method cannot accurately demonstrate the difference of the communication strength between the residues and tends to depart from the actual. By means of the method of the present application, the residue interaction weighting network is established according to interaction strength between the residues, their shortest paths are calculated based on a weight of edge between the residues, and then the important communication path in the protein is mined. Therefore, the method of the present application is closer to the actual, further demonstrates characteristics and difference of communication between the residues and is more beneficial for exploring difference between different proteins in next steps.

Part of steps in the example of the present disclosure herein may be realized through software, and a corresponding software program may be stored in a readable storage medium, such as a compact disc or a hard disk.

The foregoing descriptions are merely preferred examples of the present disclosure herein, but are not intended to limit the present disclosure herein. Any modification, equivalent substitution, improvement and the like made within the spirit and principle of the present disclosure herein shall fall within the protection scope of the present disclosure herein.

What is claimed is:

1. A method for analyzing a relationship between a communication path and heat resistance of a lipase in a dynamic residue interaction network, comprising:
working out, based on the residue interaction network, a dynamic shortest communication path between residues through a Dijkstra algorithm, and then performing frequent path mining on the shortest communication path through an Apriori-like algorithm and therefore obtaining a rigid communication path of the lipase and a relationship between the rigid communication path and thermal stability of the lipase, wherein directed sequentiality of the path and a topological characteristic of the network are considered while performing frequent path mining on the shortest communication path through the Apriori-like algorithm.

2. The method according to claim 1, comprising:
S1: extracting a three-dimensional structure of the lipase and its mutant from a PDB, and performing molecular dynamics simulation based on an Amber force field and therefore obtaining a molecular motion trajectory file of the lipase within simulation time at different temperatures; and intercepting N frames of snapshots within the simulation time, wherein N is an integer;
S2: establishing N frames of weighting residue interaction networks according to the molecular motion trajectory file obtained in the S1;
S3: calculating a shortest path between an initial residue Rₛ and a terminal residue Rₜ in the N frames of weighting residue interaction networks on the basis of the Dijkstra algorithm, wherein s≠t, s, t^{∈}{1,2, . . . ,n};
S4: performing frequent path mining based on a time sequence on a shortest path between every two residues through the Apriori-like algorithm; and
S5: performing, through the Apriori-like algorithm, frequent path mining on a time sequence frequent path or path segment mined in the S4 and therefore obtaining the rigid communication path of the lipase and then obtain the relationship between the rigid communication path and the thermal stability of the lipase.

3. The method according to claim 2, wherein the S2 comprises:
encoding a structure of the lipase in each frame of snapshot intercepted within the simulation time in the S1 into a residue interaction network changing over time; and extracting interaction force between the residues in the molecular motion trajectory file and establishing a static weighting residue interaction network within the whole simulation time according to the interaction force between the residues in each frame of snapshot through Ring2.0.

4. The method according to claim 2, wherein the S3 comprises:
calculating the shortest path between the initial residue Rₛ and the terminal residue Rₜ (s≠t, s, t^{∈}{1,2, . . . ,n}) in each frame of residue interaction network through the Dijkstra algorithm, wherein n represents the quantity of residues in the lipase, comprising the following steps:
step 1: initializing distances between the initial residue Rₛ and all the other residues; recording a distance D (Rₛ, Rᵢ) to be a length of an edge between Rₛ and Rᵢ if a direct connection exists between the initial residue Rₛ and Rᵢ; or otherwise, recording the distance D (Rₛ, Rᵢ) to be infinitely great;
step 2: setting two sets U and V, wherein the set U is configured to sequentially store discovered residues appearing in the shortest path, and at first, the set U contains only the initial residue Rₛ, and the remaining uncertain residues are stored in the set V;
step 3: comparing D (Rₛ, Rᵢ) with D (Rₛ, Rᵢ), wherein Rᵢ and Rⱼ are the residues in V having direct connection with the initial residue Rₛ, the residue Rₖ having a shortest distance from Rₛ is transferred into the set U, and i, j, k^{∈}{1,2, . . . ,n} and i≠j;
step 4: calculating a distance between the residue Rₖ newly added into U and the residue in the set V having direct connection with Rₖ; recording a distance between Rₛ and Rₘ to be D (Rₛ, Rₖ)+D (Rₖ, Rₘ) if D (Rₛ, Rₘ)>D (Rₛ, Rₖ)+D (Rₖ, Rₘ), wherein Rₘ^{∈}V; and then putting the residue in V having the shortest distance from R₅ in U; and
step 5: repeating the step 4 till the terminal residue Rₜ appears in the set U.

5. The method according to claim 2, wherein the S4 comprises:
performing frequent path mining on the shortest path between any every two residues based on the time sequence, and defining a mined frequent path as the time sequence frequent path, wherein two characteristics of the path, namely, the directed sequentiality of the path and the topological characteristic of the network are considered emphatically while mining a frequent path sequence through the Apriori-like algorithm; and
performing time sequence frequent path mining on the shortest path of all residue pairs within the simulation time on the basis of the Apriori-like algorithm, comprising the following steps:
step 1: establishing a matrix-based data model: storing the shortest path between the same pair of residues Rₛ and Rₜ at different moments according to a time sequence, sequentially recording the residues in each path to be 1, 2, 3, . . . i according to a signal transmission sequence; marking with 0 if there is no residue passed by; and only needing to judge whether a value to which communication residues correspond is an arithmetic progression with a tolerance being 1 when whether rows in a matrix contain a path sequence is judged, wherein the communication residues are the residues contained in each path;
step 2: generating a candidate frequent 1-item set, wherein a residue in each item set in a candidate frequent path set C₁ is a starting point of a candidate frequent path; and calculating a support degree Sup of each item set in C₁, and the candidate frequent path is regarded as a frequent path and stored in a set L₁ if Sup is larger than a minimum support threshold value, wherein a calculation method of the support degree Sup is as follows:
$$S u p = \frac{count}{T} ,$$
wherein count represents the number of times of appearance of a path segment between Rₛ and Rₜ within the simulation time under observation, and T represents the simulation time;
step 3: connecting the residues in L₁ mutually and therefore generating a candidate frequent 2-item set C₂, and then calculating a support degree Sup of each candidate frequent 2-item set in generated C₂, wherein the calculation method of Sup is the same as that of the step 2; and regarding that the candidate frequent 2-item set is frequent and stored in L₂ if Sup is larger than a set time minimum support degree threshold value; and
step 4: continuing the step 3 till a maximum frequent path sequence Lₚ is discovered.

6. The method according to claim 5, wherein the time minimum support degree threshold value is 0.5.

7. The method according to claim 5, wherein in the step 4, the quantity of candidate sets is reduced by using an Apriori attribute, namely, all nonempty subsequences of any frequent sequence set are frequent necessarily, otherwise, this candidate frequent path sequence is to be removed from Cₚ, and Cₚ is a set of candidate frequent paths with all path lengths being p-1.

8. The method according to claim 2, wherein in the S5, a space-time frequent path means a time sequence frequent path or path segment frequently appearing in paths with different starting points and end points and represents information of a shared communication path between the residues, thus, the space-time frequent path reflects communication characteristics of the residues from an overall perspective, before performing space-time frequent path mining, all time sequence frequent path p sequences of each pair of residues in the matrix are used as a data set, a space-time minimum support degree threshold value is set, the space-time frequent path is mined, and thus a relationship between the heat resistance of the lipase and signal transfer is explored, wherein p is a length of a longest time sequence frequent path and is not smaller than 4.

9. The method according to claim 8, wherein the space-time minimum support degree threshold value is 0.2.

10. Application of the method for analyzing a relationship between a communication path and heat resistance of lipase in a dynamic residue interaction network according to claim 1 in a field of protein structure and function.
